(19) United States
(12) Patent Application Publication
(10) Pub. No.: US 2025/0375607 A1
(43) Pub. Date: Dec. 11, 2025

(54) SAFETY MECHANISM, SAFETY SYSTEM, VAD SYSTEM AND METHOD FOR RELEASING TWO UNITS COUPLED BY A CORRESPONDING SAFETY SYSTEM

(71) Applicant: Berlin Heart GmbH, Berlin (DE)

(72) Inventor: Eike Schärff, Berlin (DE)

(73) Assignee: Berlin Heart GmbH, Berlin (DE)

(21) Appl. No.: 18/878,404

(22) PCT Filed: Jun. 21, 2023
(86) PCT No.: PCT/EP2023/066781

§ 371 Date: Dec. 23, 2024

(30) Foreign Application Priority Data
Jun. 27, 2022 (EP) 22181301.7

(51) Int. Cl. A61M 60/855

(52) U.S. Cl. CPC A61M 60/855

(57) ABSTRACT

A safety mechanism for locking and releasing an accumulator unit of a VAD system with a control unit of a VAD system may be provided. Safety mechanisms may comprise a latching hook having a latching lug, a latching opening, and an actuating element, and may have a rest position, a pre-release position, and a release position as described herein. An actuating element may be configured such that actuation of the actuating element moves the latching hook and/or the latching opening from a pre-release position into a release position.

### CROSS-REFERENCE TO RELATED APPLICATIONS

This application is a U.S. national stage application under 35 U.S.C. § 371 of PCT Application No. PCT/EP2023/066781, filed on Jun. 21, 2023, and published on Jan. 4, 2024 as WO 2024/002814 A1, which claims priority to European Patent Office Application No. EP 22181301.7, filed on Jun. 27, 2022. The entire contents of each of the above applications is incorporated herein by reference.

### BRIEF DESCRIPTION OF THE DRAWINGS

Exemplary embodiments of the invention are explained in greater detail in the figures. In the drawings

FIG. 1a shows a control unit and an accumulator unit of a VAD system in perspective view, wherein the control unit and the accumulator unit are locked together,

FIG. 1b shows the control unit and the accumulator unit of FIG. 1a in perspective view, wherein the control unit and the accumulator unit are released from each other,

FIG. 1c shows the control unit and the accumulator unit of FIGS. 1a and 1b in a plan view in a rest position,

FIG. 1d shows the control unit and the accumulator unit of FIG. 1c in a sectional view along the section AA shown in FIG. 1c in the rest position,

FIG. 1e shows the control unit and the accumulator unit of FIGS. 1a to 1d in a plan view in a pre-release position,

FIG. 1f shows the control unit and the accumulator unit of FIGS. 1a-1e in a sectional view along the section AA shown in FIG. 1e in the pre-release position,

FIG. 1g shows a detailed view of the actuating element and the latching hook of the previous figures in the release position,

FIG. 1h shows the control unit and the accumulator unit of FIGS. 1a-1g in a sectional view along the section AA shown in FIG. 1e in the release position,

FIG. 1i shows a VAD system in the rest position, which is configured according to the VAD system of FIGS. 1a to 1f, wherein an elastomer is provided as an elastic element between the first and the second unit instead of a spring,

FIG. 1j shows a VAD system in the pre-release position, which is configured according to the VAD system of FIGS. 1a to 1f, wherein an elastomer is provided as an elastic element between the first and the second unit instead of a spring,

FIG. 2a shows a VAD system in perspective view, wherein the control unit and the accumulator unit are released from each other,

FIG. 2b shows the control unit and the accumulator unit of FIG. 2a in a plan view in a rest position, in which the control unit and the accumulator unit are locked together

FIG. 2c shows the control unit and the accumulator unit of FIG. 2b in a sectional view along the section BB shown in FIG. 2b in the rest position,

FIG. 2d shows the control unit and the accumulator unit of FIGS. 2b and 2c in a sectional view along the section AA shown in FIG. 2c in the rest position,

FIG. 2e shows the control unit and the accumulator unit of FIGS. 2a to 2d in a plan view in a pre-release position,

FIG. 2f shows the control unit and the accumulator unit of FIG. 2e in a sectional view along the section BB shown in FIG. 2e in the pre-release position,

FIG. 2g shows the control unit and the accumulator unit of FIGS. 2e and 2f in a sectional view along the section AA shown in FIG. 2f in the pre-release position,

FIG. 2h-j show a VAD system which is configured in accordance with the VAD system of FIGS. 2a to 2g, wherein an elastomer is provided as an elastic element between the first and the second unit instead of a spring, wherein 2h shows a section AA,

FIG. 2i a section BB and FIG. 2j a plan view,

FIG. 3a shows a VAD system in perspective view, wherein the control unit and the accumulator unit are released from each other,

FIG. 3b shows the control unit and the accumulator unit of FIG. 3a in a plan view in a rest position, in which the control unit and the accumulator unit are locked together,

FIG. 3c shows the control unit and the accumulator unit of FIG. 3b in a sectional view along the section AA shown in FIG. 3b in the rest position,

FIG. 3d shows the control unit and the accumulator unit of FIGS. 2b and 2c in a sectional view along the section BB shown in FIG. 3c in the rest position,

FIG. 3e shows the control unit and the accumulator unit of FIGS. 3a to 3d in a plan view in a release position,

FIG. 3f shows the control unit and the accumulator unit of FIG. 3e in a sectional view along the section AA shown in FIG. 3e in the release position,

FIG. 3g shows the control unit and the accumulator unit of FIG. 3f in a sectional view along the section BB shown in FIG. 3f in the release position,

FIG. 3h-j show a VAD system which is configured in accordance with the VAD system of FIGS. 3a to 3g, wherein an elastomer is provided as an elastic element between the first and the second unit instead of a spring, wherein 3h shows a section BB,

FIG. 3i a section AA and FIG. 2j a plan view.

Recurring features are provided with the same reference signs in the figures.

### DETAILED DESCRIPTION

The present disclosure relates to a safety mechanism for locking and releasing a first unit with a second unit, in particular for locking and releasing an accumulator unit of a VAD system with a control unit of a VAD system. A VAD system is a cardiac support system, with VAD standing for Ventricular Assist Device. Furthermore, the present disclosure relates to a safety system and a VAD system, each comprising a corresponding safety mechanism. The present disclosure also relates to a method for releasing a corresponding safety mechanism.

Cardiac support systems are known in the prior art. A ventricular assist device (VAD) is an electromechanical cardiovascular support device configured to either partially or completely replace the function of a failing heart. VAD systems typically comprise an implantable heart pump, a control unit that controls the heart pump and a battery or accumulator unit to supply the control unit and/or the heart pump with power. The control unit and the accumulator or battery unit are typically arranged outside the patient's body. In order to ensure that the VAD system functions properly, a reliable power supply must be ensured. The accumulator unit or battery unit must therefore be reliably attached to the control unit. To change batteries or accumulators, the accumulator or battery unit must be releasable from the control unit. So as not to jeopardize the power supply to the heart pump and/or the control unit, it must be ensured that the accumulator or battery unit and the control unit are not accidentally disconnected from each other.

The object of the present invention is therefore to propose a secure and releasable connection between an accumulator or battery unit and a control unit, in particular for a VAD system.

This object is achieved by a safety mechanism and/or a safety system and/or a VAD system and/or a method. Advantageous developments of the invention are described in this application.

The figures appended to this disclosure and the accompanying description of the figures serve to illustrate exemplary embodiments of the invention and are not intended to be restrictive. In particular, the features and combinations of features described there are not mandatory and are merely examples.

The proposed safety mechanism is used to lock and release a first unit with a second unit. In particular, the proposed safety mechanism is used to releasably lock an accumulator unit of a VAD system to a control unit of a VAD system.

In this description, the term “accumulator unit” is used, which according to the wording may include an accumulator. However, this term is not intended to be restrictive. The accumulator unit may therefore additionally or alternatively comprise a battery.

The safety mechanism comprises a latching hook having a latching lug, a latching opening and a first actuating element. The latching hook may be moved between a rest position, a pre-release position and a release position. To do this, the latching hook may be actively moved from one position to the other by moving the latching hook. Alternatively or additionally, another component, for example the latching opening, may be moved so that the latching hook is moved between the rest position, the pre-release position and the release position.

In the rest position the latching hook engages in the latching opening in such a way that the latching hook projects through the latching opening and the latching lug engages at least partially behind an edge of the latching opening.

In the pre-release position the latching hook engages at least partially in the latching opening in such a way that at least part of the latching hook projects through the latching opening and the latching lug is released from the edge of the latching opening.

In the release position, the latching hook is arranged outside the latching opening.

The actuating element is configured here in such a way that actuation of the actuating element moves the latching hook and/or the latching opening from the pre-release position into the release position.

This may have the advantage that the latching hook may only be released from the latching opening by additional actuation, so that unintentional release of the latching hook from the latching opening may be avoided as far as possible.

In an advantageous embodiment the latching hook and the latching opening may be configured in such a way that in the rest position the latching hook is locked with the latching opening in a first direction and in a second direction, and the latching hook is movable in a direction for bringing it into the pre-release position. In particular, the second direction may be perpendicular to the first direction.

In particular, the second direction may correspond to an actuating direction of the actuating element. For example, pressing a push button may be disabled. The direction of movement into the pre-release position may be perpendicular to the first direction and/or the second direction. In particular, the direction of movement into the pre-release position may displace the latching hook along its longitudinal axis and/or the latching opening. In the rest position, for example, the latching hook may only be moved a predefined distance along its longitudinal axis. For example, the latching hook may only be movable into the pre-release position. In particular, the latching hook may be unable to be moved directly from the rest position into the release position, i.e., in particular without it having been moved into the pre-release position beforehand and without the actuating element having been actuated in the pre-release position.

For example, it may be provided that the latching hook may only be brought into the pre-release position by a user by a defined movement, in particular by a movement of the first unit in the direction of the second unit.

The latching hook in the pre-release position may be arranged in such a way that it engages in the latching opening in such a way that the latching hook is locked with the latching opening in the first direction and is released in the second direction. In this way, it may be provided that the latching hook may not be released from the latching opening along the first direction. However, in the pre-release position, the actuating element may be released in such a way that it may be actuated.

The actuating element may be configured in such a way that actuation is blocked when the latching hook is in the rest position. The actuation of the actuating element may, for example, be blocked in the rest position by the latching lug. In this way, accidental actuation of the actuating element may be avoided as far as possible.

The actuating element may be configured as a pressure element, for example. In particular it may be configured in such a way that a pressure force exerted on the actuating element moves the latching hook and/or the latching opening in such a way that the latching hook is brought from the pre-release position into the release position. In the release position, the unit on which the latching hook is arranged and the unit on which the latching opening is arranged may be separated from each other. The steps described above, which must be carried out in order to separate the unit on which the latching hook is arranged from the unit on which the latching opening is arranged, i.e., to move it from the rest position to the pre-release position and from there to the release position, may increase the safety of the safety mechanism.

In a preferred embodiment, the actuating element is fixedly connected to the latching opening. Actuation of the actuating element (which is only possible in particular when the latching hook is in the pre-release position) may displace the latching opening along the second direction. Displacing the latching opening may cause the latching hook to move out of the latching opening. This allows the latching hook to be brought into the release position after the actuating element has been actuated. Alternatively, the latching hook may be fixedly connected to the actuating element. Actuation of the actuating element (which is particularly only possible when the latching hook is in the pre-release position) may then move the latching hook in such a way that it is moved out of the latching opening.

The latching hook and/or the latching opening may, for example, be made of plastic and/or may comprise plastic. In particular, the latching hook and/or the latching opening may be made of a material that is at least partially elastically bendable. For example, actuation of the actuating element may elastically bend the latching hook and/or the latching opening in such a way that the latching hook is moved out of the latching opening or the latching opening is moved away from the latching hook. If the actuating element is no longer actuated, the latching hook or the latching opening may return elastically to its original position.

In a preferred embodiment, the latching hook and the latching opening may be released as described below, wherein in the initial position the latching hook is locked in the rest position with the latching opening. When the latching hook is engaged with the latching opening in the rest position, the first and second units are typically locked together The latching hook may be brought from the rest position into the pre-release position. The actuating element may be actuated in the pre-release position. While the actuating element continues to be actuated, the first unit may be released from the second unit.

Actuation of the actuating element is blocked in the rest position. Actuation of the actuating element may be blocked by a form fit. To block, for example, the latching lug of the latching hook may engage behind an edge of the latching opening.

In the rest position, the latching hook and the latching opening may be moved into the pre-release position. The latching opening may, for example, be configured as a slot into which the latching hook can be displaced. The cross-section of a part of the latching hook that protrudes through the latching opening may therefore be smaller than the latching opening in particular. For example, a clearance may be provided between the latching hook and the latching opening. A relative movement of the first unit and the second unit may cause the latching hook to displace in the latching opening. The shape and size of the latching opening may limit the movement of the latching hook in the latching opening. To move the latching hook in the latching opening, the first unit may be moved in the direction of the second unit and/or the second unit may be moved in the direction of the first unit, for example. In particular, this movement may run along a longitudinal axis of the latching hook. In the rest position, the latching hook may be arranged at a first position in the latching opening configured as a slot and in the pre-release position the latching hook may be arranged at a second position in the latching opening configured as a slot. The slot may be configured in such a way that the latching hook may be movable at least 1 mm, preferably at least 2 mm, particularly preferably at least 3 mm in the latching opening when the latching hook is in the rest position. The slot may be configured in such a way that the latching hook may be movable a maximum of 6 mm, preferably a maximum of 5 mm, particularly preferably a maximum of 4 mm in the latching opening when the latching hook is in the rest position.

Actuation of the actuating element may be released in the pre-release position. In particular, the latching opening and/or the latching hook and/or the latching lug may be configured in such a way that in the pre-release position the latching lug does not engage behind the edge of the latching opening. In the pre-release position, the latching hook may partially protrude through the latching opening. The latching lug may be located here below or above the latching opening but within the edge of the latching opening in such a way that actuation of the actuating element, for example pressing in, is released. The actuation of the actuating element may, for example, move the latching opening in such a way that the latching opening is moved away from the latching hook so that the latching hook is in the release position. Alternatively, the actuation of the actuating element may, for example, move the latching hook in such a way that the latching hook is moved out of the latching opening so that the latching hook is in the release position. A first elastic element, for example a spring, may be arranged on the actuating element to push the actuating element into an initial position in which the latching opening and the latching hook are locked together. To actuate the actuating element, it may therefore be provided that a force must be exerted on the actuating element that is greater than an elastic force of the first elastic element, which pushes the actuating element into the initial position. If the first elastic element is a spring, for example, the force required to actuate the actuating element must be greater than the spring force. Alternatively or in addition to a first elastic element, the actuating element itself may comprise an elastic material. The actuation of the actuating element may then, for example, bend the latching opening or the element that comprises the latching opening in such a way that the latching opening is bent away from the latching hook so that the latching hook is in the release position. In particular, the actuating element may be integral with the latching opening. Alternatively, the actuation of the actuating element may, for example, bend the latching hook or an element that comprises the latching hook in such a way that the latching hook is bent away from the latching opening so that the latching hook is in the release position. In particular, the actuating element may be integral with the latching hook.

In a preferred embodiment, it is provided that—in order to release the first unit from the second unit—the first unit and the second unit are pulled apart when the latching hook is in the release position. In particular, it may be provided that the first unit and the second unit, or the latching hook and the latching opening, must be pulled apart in a direction along or parallel to the longitudinal axis of the latching hook. In particular, it may be necessary for the actuating element to remain actuated so that the latching hook remains in the release position.

In a preferred embodiment the safety mechanism comprises a second elastic element which is configured and arranged in such a way that it pushes the latching hook from the pre-release position into the rest position. The second elastic element may, for example, be configured as a spring or may comprise a spring. Alternatively or additionally, the elastic element may comprise or form an elastomer, for example in the form of a rubber seal. A rubber seal may be partially arranged on the first and/or the second unit or may be formed all the way round. In particular, the rubber seal may be arranged circumferentially on a contact surface between the first and the second unit. The rubber seal may seal the first unit to the second unit. A rubber seal may have the advantage that, in addition to its elastic property, it may seal a housing interior, which is arranged in the first and/or second unit, for example in a watertight and/or gas-tight and/or dust-tight manner, as long as the first and second units are connected. The rubber seal may also have substantially no or little sealing properties and may merely serve as an elastic connection between the first and second unit.

In one embodiment, the safety mechanism comprises a second latching hook, a second latching opening and a second actuating element. The second latching hook may be configured and/or arranged mirror-symmetrically to the first latching hook. The second latching opening may be configured and/or arranged mirror-symmetrically to the second latching opening. The second actuating element may be configured and/or arranged mirror-symmetrically to the first actuating element.

The features described above with regard to the first latching hook and/or the first latching opening and/or the first actuating element may also be applied to the second latching hook and/or the second latching opening and/or the second actuating element.

In a preferred embodiment, the latching hook described above is arranged on a first side of the first unit and a latching hook of the same configuration is arranged mirror-symmetrically on a second side of the first unit. In this embodiment, the latching opening described above is arranged on the second unit on a first side of the first unit and a latching opening of the same configuration is arranged mirror-symmetrically on a second side of the second unit. This has the advantage that releasing the first unit from the second unit requires actuation, in particular simultaneous actuation, of the first and second actuating elements. This may make the safety mechanism safer, as unintended release is less likely.

The first and second actuating elements may be arranged opposite each other. In particular, the first and second actuating elements may be arranged opposite each other in such a way that simultaneously pressing the actuating elements together causes the first and second latching hooks to be in the release position. In particular, the first and second actuating elements may be arranged opposite each other in such a way that the distance between the actuating elements is a maximum of 7 cm, preferably a maximum of 6 cm, particularly preferably a maximum of 4 cm. In particular, the first and second actuating elements may be arranged opposite each other in such a way that the distance between the actuating elements is a minimum of 1 cm, preferably a minimum of 2 cm, particularly preferably a minimum of 3 cm. In particular, the first and second actuating elements may be arranged opposite each other in such a way that an adult user may actuate the first and second actuating elements with one hand, i.e., with the right hand or the left hand, for example with a thumb and an index finger. In particular, a distance may be selected so that a hand size from the 5th percentile up to the 95th percentile of the users to be deployed is sufficient to operate the first and the second actuating elements simultaneously, in particular in accordance with DIN 33402-2:2005-12.

In particular, the first and second actuating elements may be arranged opposite each other in such a way that a child of 6 years of age may actuate the first and second actuating elements with one hand, for example with a thumb and an index finger. In particular, a maximum distance may be greater than 2 cm, preferably greater than 3 cm, particularly preferably greater than 3.5 cm. In particular, a maximum distance may be less than 8 cm, preferably less than 6 cm, particularly preferably less than 5 cm. In particular, the first and second actuating elements may be arranged opposite each other in such a way that a child of 3 years or younger is unable to actuate the first and second actuating elements with one hand, for example with a thumb and an index finger.

A further safety element may be provided to prevent accidental release of the first unit from the second unit. The safety element may be able to be brought from a safety position into a release position. In the safety position, the safety element may block actuation of the actuating element. In the release position, the safety element may release actuation of the actuating element. In this exemplary embodiment, to release the first unit from the second unit, the latching hook would first have to be moved from the rest position to the pre-release position (as in the exemplary embodiments above). In the pre-release position, the safety element would then have to be brought from the safety position into the release position so that the actuating element may be actuated. If a first and a second actuating element are provided, the safety element may simultaneously block the first and the second actuating element in the safety position. Bringing the safety element into the release position may release the first and second actuating elements simultaneously. Alternatively, a first safety element may be provided that blocks the first actuating element in the safety position and a second safety element may be provided that blocks the second actuating element in the safety position. To actuate the first and second actuating element, both the first and second safety element would then have to be brought from the safety position into the release position. In the release position, the (first and/or second) actuating element may then be actuated in order to release the first unit from the second unit.

The present disclosure further relates to a safety system comprising the first unit and the second unit, wherein the first unit is releasably connectable to the second unit via the safety mechanism described above. In particular, the latching hook is fixedly connected to the first unit and/or is part of the first unit. The latching opening may be fixedly connected to the second unit and/or may be part of the second unit. The latching hook may be integral with the first unit and/or welded, clamped and/or screwed to the first unit. The latching opening may be integral with the second unit and/or welded, clamped and/or screwed to the second unit.

In particular, the first unit and the second unit have an electrical connection that may be disconnected. In particular, the electrical connection is established when the first and second units are locked with one another. In particular, the electrical connection is established when the first and second units, or the latching hook and the latching opening, are in the rest position and/or in the pre-release position. The electrical connection may comprise a plug and a socket. In particular, the plug may be arranged on the first unit and the socket may be arranged on the second unit or vice versa. If the first and second units are an accumulator unit and a control unit and these are locked with one another, the control unit is supplied with power in particular from the accumulator or the battery of the accumulator unit.

In an advantageous embodiment, the first unit is a plastics injection-molded part and the latching hook is formed in one piece with the first unit. In an advantageous configuration, the second unit is a plastics injection-molded part and the latching opening is formed in one piece with the first unit. For example, the first unit and/or the second unit may comprise or be made of one or more of the following materials: polypropylene (PP) and/or acrylonitrite butadiene styrene (ABS) and/or polycarbonate (PC) and/or polymethyl methacrylate (PMMA) and/or aliphatic polyamides (PPA) and/or polyoxymethylene (POM) and/or polybutylene terephthalate (PBT) and/or polyetherimide (PEI) and/or polyethylene (PE).

In particular the first unit and the second unit are locked together in the rest position and in the pre-release position via the safety mechanism and are released from each other in the release position.

The present disclosure further relates to a VAD system comprising a safety system as described above. The VAD system may also comprise a heart pump. The first unit may be a control unit. The second unit may be a battery unit or an accumulator unit. Alternatively, the first unit may be a battery unit or an accumulator unit and the second unit may be a control unit.

Furthermore, the present disclosure relates to a method for releasing the first unit from the second unit of the system described above, in particular the VAD system described above.

The method comprises the steps of
I. “moving the first unit in the direction of the second unit”
II. actuating the actuating element
III. releasing the first unit from the second unit,
   wherein step II follows step I and step III follows step II to release the first unit from the second unit.

In one variant of the method, a safety element may be brought from a safety position into a release position between steps I and II. The safety element may block actuation of the actuating element in the safety position and release actuation of the actuating element in the release position.

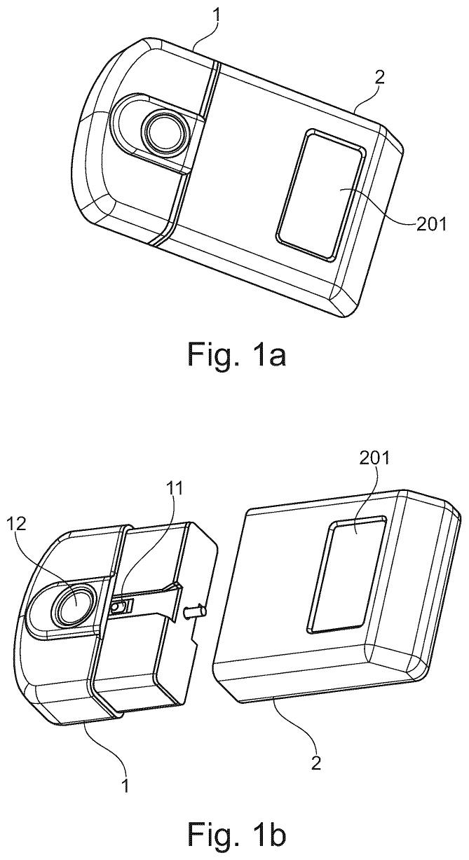

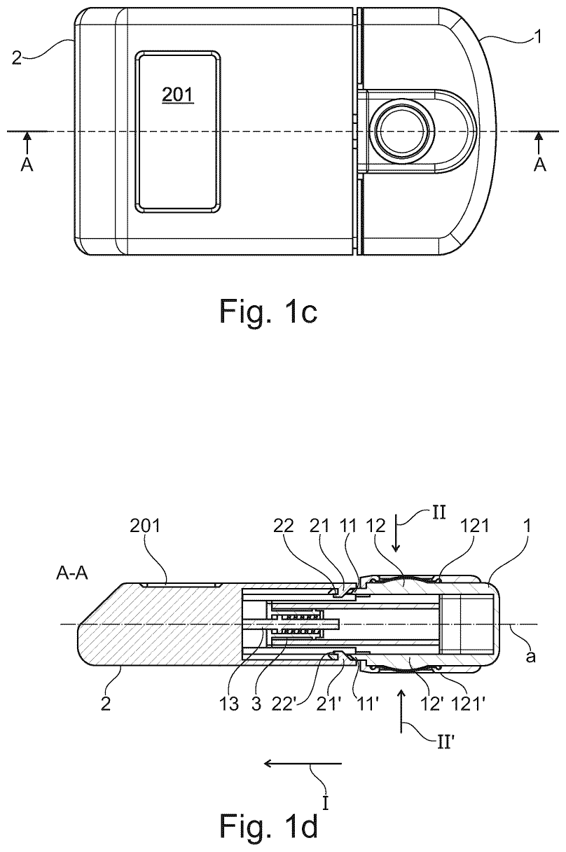

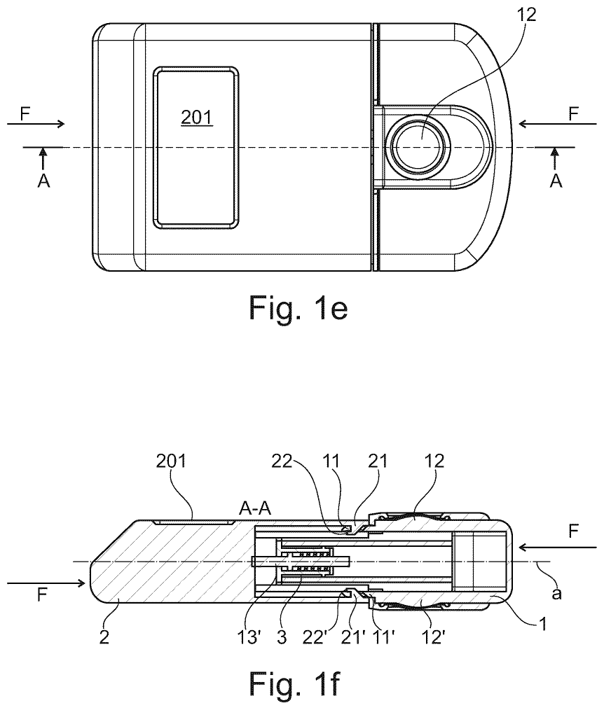

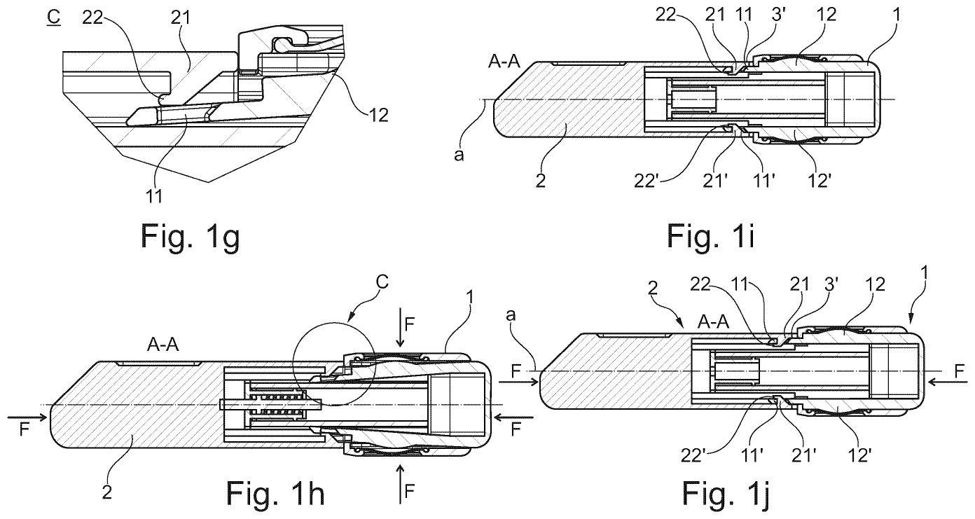

FIG. 1a shows a control unit 2 and an accumulator unit 1 of a VAD system in a perspective view. The accumulator unit 1 comprises an accumulator for supplying the control unit 2 with power. The control unit 2 comprises an electronic device, for example a control unit. The control unit is set up to control a heart pump. The heart pump (not shown) is connected to the control unit wirelessly or by cable. The control unit 2 comprises a display 201 for displaying various information items, for example for displaying an accumulator charge of the accumulator of the accumulator unit 1 and/or for displaying heart data and/or for displaying data of the heart pump.

The accumulator unit 1 and the control unit 2 are releasably locked together via a safety mechanism. FIG. 1a shows the VAD system in a rest position. FIG. 1b shows the accumulator unit 1 and the control unit 2 in a position released from one another. The safety mechanism for releasably locking the accumulator unit 1 to the control unit 2 comprises a latching hook 21, a latching opening 11 and a first actuating element 12. The actuating element 12 and the latching opening 11 are part of the accumulator unit 1. The latching hook 21 is part of the control unit 2.

FIG. 1c shows a plan view of the accumulator unit 1 and the control unit 2 in a locked rest position. FIG. 1d shows the accumulator unit 1 and the control unit 2 in a locked rest position in a sectional view along the section AA shown in FIG. 1c. The latching hook 21 is integral with the housing of the control unit 2. The latching hook has a latching lug 22. The actuating element 12 is integral with the housing of the accumulator unit 1 and integral with the latching opening 11. In the rest position shown in FIG. 1c and FIG. 1d, the latching hook 21 protrudes through the latching opening 11. The latching lug 22 engages behind an edge of the latching opening 11 so that actuation of the actuating element 12 is blocked in a direction II.

Mirror-symmetrically to an axis a, the accumulator unit 1 has a second actuating element 12′ and a second latching opening 11′, which are formed integrally with the housing of the accumulator unit 1. The control unit 2 likewise has a second latching hook 21′ with a second latching lug 22′ mirror symmetrically to the axis a. The latching hook 21′ is integral with the housing of the control unit 2. In the rest position shown in FIG. 1d, the latching hook 21′ protrudes through the latching opening 11′. The latching lug 22′ engages behind an edge of the latching opening 11′ so that actuation of the actuating element 12′ in a direction II′ is blocked.

A spring 3 is arranged in the accumulator unit 1. In the rest position, the spring 3 pushes the control unit 2 along the axis a away from the accumulator unit 1. In the exemplary embodiment shown here, the spring 3 is a coil spring. The accumulator unit comprises a pin 13, which is connected to the housing of the accumulator unit 1 so that the pin may move in translation. The pin 13 is pushed along the axis a in direction I by the spring 3. The pin 13 may comprise an electrical connection between the accumulator or the battery arranged in the accumulator unit 1 and the control unit 2. Alternatively, a plug and a socket of a different shape may be provided for the electrical connection between the accumulator or the battery of the accumulator unit 1 and the control unit 2. The plug and the socket may be configured in such a way that the elastic element pushes them apart. When the accumulator unit 1 and the control unit are assembled, the pin 13 touches the control unit 2. To assemble the accumulator unit 1 and the control unit 2, the accumulator unit 1 and the control unit 2 are pressed together, wherein the pressure force is greater than the spring force of the spring 3. In the rest position, i.e. in the state shown in FIGS. 1c and 1d, the spring 3 exerts a force on the control unit 2. The spring 3 is therefore preloaded in the rest position. If no external pressure force is exerted on the accumulator unit 1 and/or the control unit 2, the spring 3 pushes the accumulator unit 1 and the control unit apart along the axis a. The latching hook is pushed into the position shown in FIG. 1d in the latching opening 11, which is configured as a slot. In this rest position, the latching lug 22 engages behind the edge of the latching opening 11 and the latching lug 22′ engages behind the edge of the latching opening 11′.

The actuating element 12 has an elastic diaphragm 121 which is arranged on an actuating surface of the actuating element 12. The elastic diaphragm 121 serves as a seal and also improves the feel of the actuating element 12. The actuating element 12′ has an elastic diaphragm 121′, which is arranged on an actuating surface of the actuating element 12′. The elastic diaphragm 121′ serves as a seal and also improves the feel of the actuating element 12′. The interior of the accumulator unit may be sealed in a watertight, gas-tight and/or dust-tight manner.

FIG. 1e shows the same view as in FIG. 1c and FIG. 1f shows the same sectional view as in FIG. 1d. In FIGS. 1e and 1f, however, the control unit 1 and the accumulator unit 1 are shown in a pre-release position. Starting from the rest position shown in FIGS. 1c and 1d, the accumulator unit 1 was moved in the direction of the control unit 2 along the axis a by 3 mm. In the pre-release position, the latching hook 21 continues to protrude through the latching opening 11 and the latching hook 21′ continues to protrude through the latching opening 11′. However, the latching lug 22 no longer engages behind the edge of the latching opening 11 and the latching lug 22′ no longer engages behind the edge of the latching opening 11′. Compared to the rest position shown in FIG. 1d, the control unit in FIG. 1f is moved 3 mm in the opposite direction I along the axis a towards the accumulator unit 1. A force F acts on the control unit 2 that is greater than the spring force of the spring 3, which pushes the accumulator unit 1 and the control unit 2 apart.

FIG. 1h shows the control unit 2 and the accumulator unit 1 of the previous figures in section AA in a release position. A detail C is marked in FIG. 1h and is shown in enlarged form in FIG. 1g. Accordingly, FIG. 1g shows a schematic representation of the actuating element 12, the latching opening 11 and the latching hook 22 of FIGS. 1a-1h. FIGS. 1h and 1g show the actuating element 12, the latching opening 11 and the latching hook 22 in the release position. A force F acts on the actuating element 12 and deflects the actuating element 12 downwards together with the latching opening 11. This releases the latching hook 21. If the actuating element 12′ is also actuated at the same time, the accumulator unit 1 may be released from the control unit 2. For the actuating element 12 and 12′ to be actuated, the accumulator unit 1 and the control unit 2 must be brought into the pre-release position (as shown in FIGS. 1e and 1f), as otherwise the latching lug 22 or 22′ will block any deflection of the actuating element 12 or 12′. The actuating element 12 and the latching opening 11 as well as the actuating element 12′ and the latching opening 11′ are made of plastic, in particular PC and/or ABS and/or polyphthalamide (PPA). The deformation of the actuating element 12 (as well as the deformation of the actuating element 12′) into the release position shown in FIG. 1g or 1h is elastic, so that the actuating element 12 (or the actuating element 12′) moves back into the position shown in FIG. 1f as soon as force F is no longer exerted on the actuating element 12 (or 12′).

FIGS. 1i and 1j show a sectional view AA through a control unit 2 and an accumulator unit 1, which corresponds to those of FIGS. 1a-g, wherein instead of a spring 3, an elastic element 3′ in the form of a circumferential elastic sealing element, for example a sponge rubber ring, is arranged between the control unit 2 and the accumulator unit 1. The features described above in relation to the previous figures therefore also apply to the VAD system shown in FIGS. 1i and 1j. FIG. 1i shows the control unit 2 and the accumulator unit 1 in an assembled and locked state in the rest position. FIG. 1j shows the control unit 2 and the accumulator unit 1 in the pre-release position. In the pre-release position, the circumferential elastic sealing element is compressed by a force F that acts on the accumulator unit. The latching lug 22 does not engage behind the edge of the latching opening 11 in FIG. 1j.

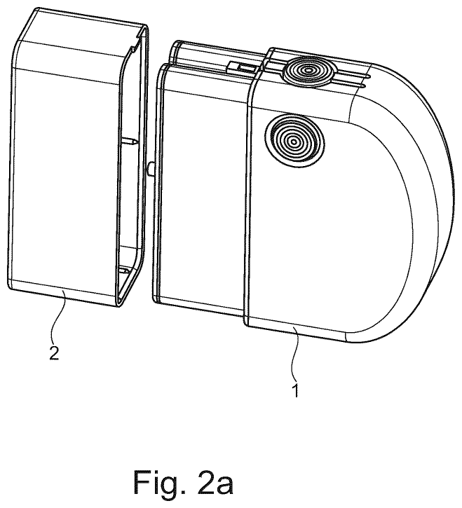

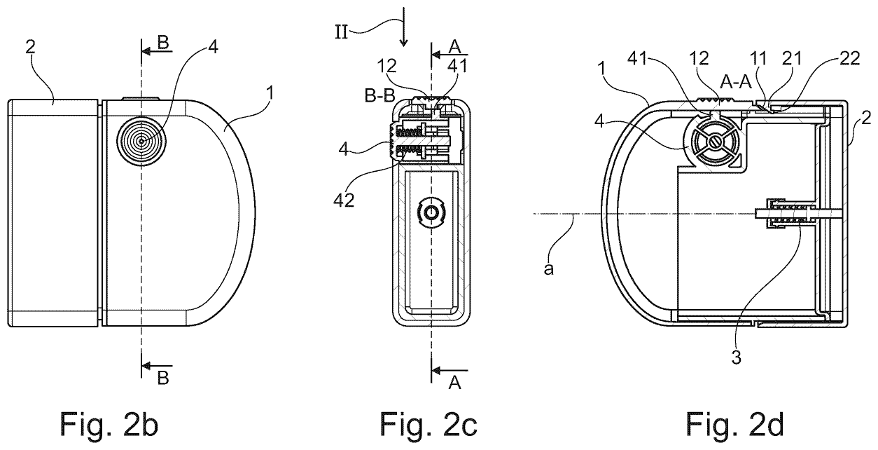

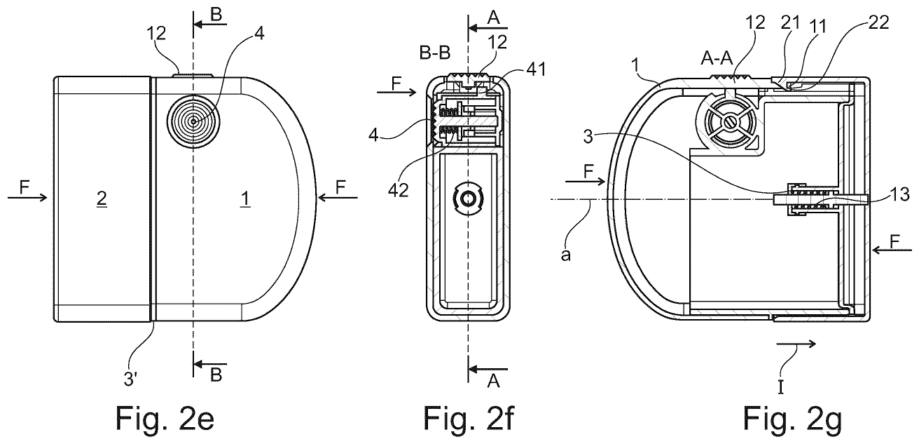

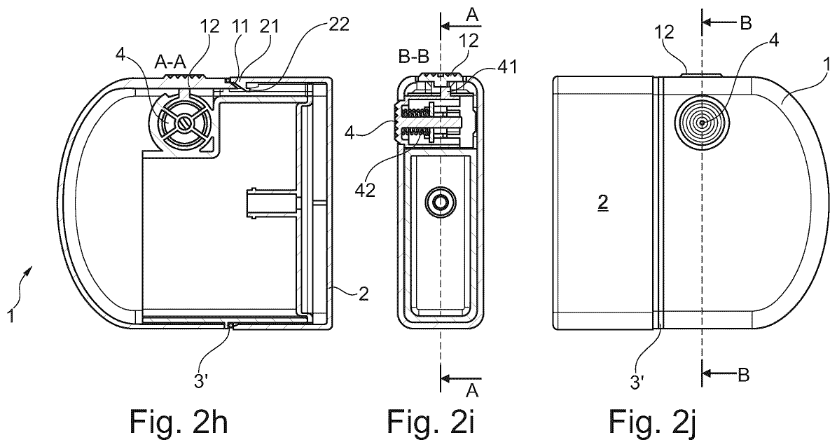

FIGS. 2a-j show a VAD system with an alternative embodiment of a safety mechanism for releasably locking an accumulator unit 1 to a control unit 2. FIG. 2a shows a perspective view of the VAD system, wherein the accumulator unit 1 and the control unit 2 are released from each other.

The accumulator unit 1 comprises an accumulator to supply the control unit 2 with power. The control unit 2 comprises an electronic device, for example a control unit. The control unit is set up to control a heart pump. The heart pump (not shown) is connected to the control unit wirelessly or by cable. The control unit 2 may comprise a display (not shown) to display various information items, for example to display an accumulator charge of the accumulator of the accumulator unit 1 and/or to display heart data and/or to display data of the heart pump.

The accumulator unit 1 and the control unit 2 are releasably locked together via a safety mechanism. FIGS. 2b to 2d show the VAD system in a rest position. FIGS. 2e to 2g show the VAD system in a pre-release position. The safety mechanism for releasably locking the accumulator unit 1 to the control unit 2 comprises a latching hook 21, a latching opening 11 and an actuating element 12. The actuating element 12 and the latching opening 11 are part of the accumulator unit 1. The latching hook 21 is part of the control unit 2.

FIG. 2b shows a plan view of the accumulator unit 1 and the control unit 2 in a locked rest position. FIG. 2c shows a section through the accumulator unit 1 along the section BB shown in FIG. 2b. FIG. 2d shows the accumulator unit 1 and the control unit 2 in a locked rest position in a sectional view along the section AA shown in FIG. 2c. FIGS. 2e to 2g show a plan view of the accumulator unit 1 and the control unit 2 in a pre-release position. FIG. 2f shows a section through the accumulator unit 1 along the section BB shown in FIG. 2e. FIG. 2g shows the accumulator unit 1 and the control unit 2 in a pre-release position in a sectional view along the section AA shown in FIG. 2f.

The latching hook 21 is integral with the housing of the control unit 2. The latching hook has a latching lug 22. The actuating element 12 is integral with the housing of the accumulator unit 1 and integral with the latching opening 11. In the rest position shown in FIGS. 2b to 2d, the latching hook 21 protrudes through the latching opening 11. The latching lug 22 engages behind an edge of the latching opening 11 so that actuation of the actuating element 12 is blocked in a direction II.

The accumulator unit 1 has a safety element 4. The safety element 4 may be brought from a safety position into a release position. The safety element 4 blocks actuation of the actuating element 12 in the safety position and releases actuation of the actuating element 12 in the release position. The safety element 4 is configured as a push button. FIGS. 2b to 2d show the safety element 4 in the safety position. A peg 41 of the safety element 4 blocks the actuation of the actuating element 12. The safety element has a spring 42 that pushes the safety element into the safety position. To bring the safety element into the release position, a user must apply a force F to the safety element 4, which compresses the spring 42 (as shown in FIG. 2f). FIGS. 2e to 2g show the safety element 4 in the release position. A user applies a force F to the safety element so that the peg 41 of the safety element 4 releases actuation of the actuating element 12.

A spring 3 is arranged in the accumulator unit 1. In the rest position, the spring 3 pushes the control unit 2 along the axis a away from the accumulator unit 1. In the exemplary embodiment shown here, the spring 3 is a coil spring. The accumulator unit comprises a pin 13, which is connected to the housing of the accumulator unit 1 so that the pin may move in translation. The pin 13 is pushed along the axis a in direction I by the spring 3. When the accumulator unit 1 and the control unit are assembled, the pin 13 touches the control unit 2. To assemble the accumulator unit 1 and the control unit 2, the accumulator unit 1 and the control unit 2 are pressed together, wherein the pressure force is greater than the spring force of the spring 3. In the rest position, i.e. in the state shown in FIGS. 2b-2d and 1d, the spring 3 exerts a force on the control unit 2. The spring 3 is therefore preloaded in the rest position. If no external force is exerted on the accumulator unit 1 or the control unit 2, the spring 3 pushes the accumulator unit 1 and the control unit apart 2 along the axis a. The latching hook 21 is pushed into the position shown in FIG. 2d in the latching opening 11, which is configured as a slot. In this rest position, the latching lug 22 engages behind the edge of the latching opening 11.

Starting from the rest position shown in FIGS. 2b to 2d, the accumulator unit 1 and the control unit 2 were pressed together along the axis a in order to move the accumulator unit 1 or the control unit 2 into the pre-release position shown in FIGS. 2e to 2g. In the pre-release position, the latching hook 21 continues to protrude through the latching opening 11. However, the latching lug 22 no longer engages behind the edge of the latching opening 11. Compared to the rest position shown in FIG. 2d, the control unit in FIG. 2e is moved 3 mm in the opposite direction I along the axis a towards the accumulator unit 1. A force F acts on the control unit 2 that is greater than the spring force of the spring 3, which pushes the accumulator unit 1 and the control unit 2 apart.

To bring the accumulator unit 1 and control unit into the release position, the accumulator unit 1 and control unit 2 must first be pressed together so that they are in the pre-release position. The safety element 4 is then actuated and kept actuated so that actuation of the actuating element 12 is released. The actuating element 12 is then actuated, causing the latching opening to bend downwards. The latching hook 21 is now outside the latching opening 11 (cf. FIG. 1g). The accumulator unit 1 and the control unit 2 may now be released from each other. The deformation of the actuating element 12 into the release position is elastic, so that the actuating element 12 moves back into the position shown in FIGS. 2e-g as soon as force F is no longer exerted on the actuating element 12. If no more force is exerted on the safety element, it is also moved back into its initial position by a spring 42, in which it blocks actuation of the actuating element 12.

FIG. 2h shows a sectional view AA, FIG. 2i a sectional view BB and FIG. 2j a plan view of a control unit 2 and an accumulator unit 1, which correspond to those of FIGS. 2a-g, wherein an elastic element 3′ in the form of a circumferential elastic sealing element, for example a sponge rubber ring, is arranged between the control unit 2 and the accumulator unit 1 instead of a spring 3. The features described above in relation to the previous figures therefore also apply to the VAD system shown in FIGS. 2h-j. FIG. 2h-j show the control unit 2 and the accumulator unit 1 in an assembled and locked state in the rest position.

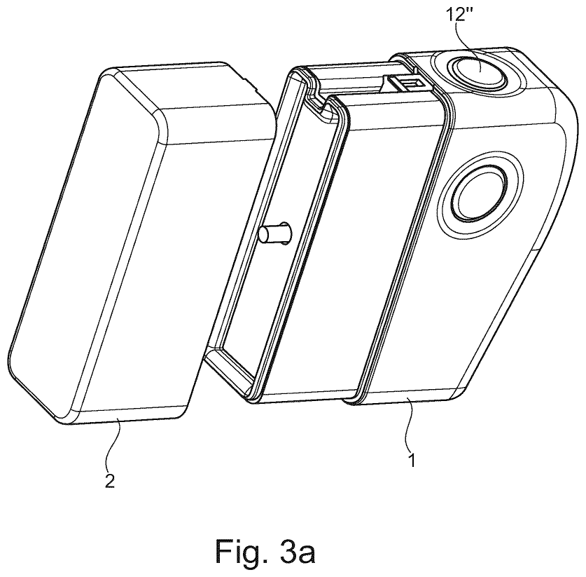

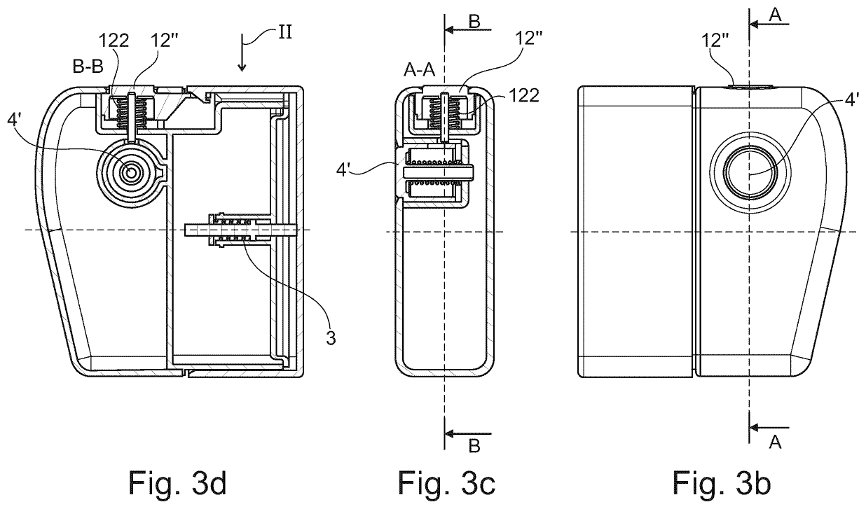

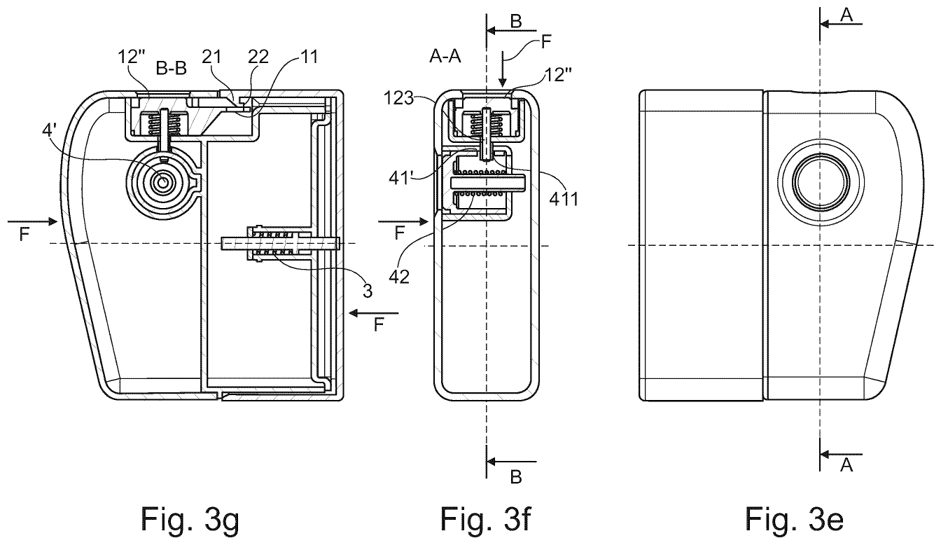

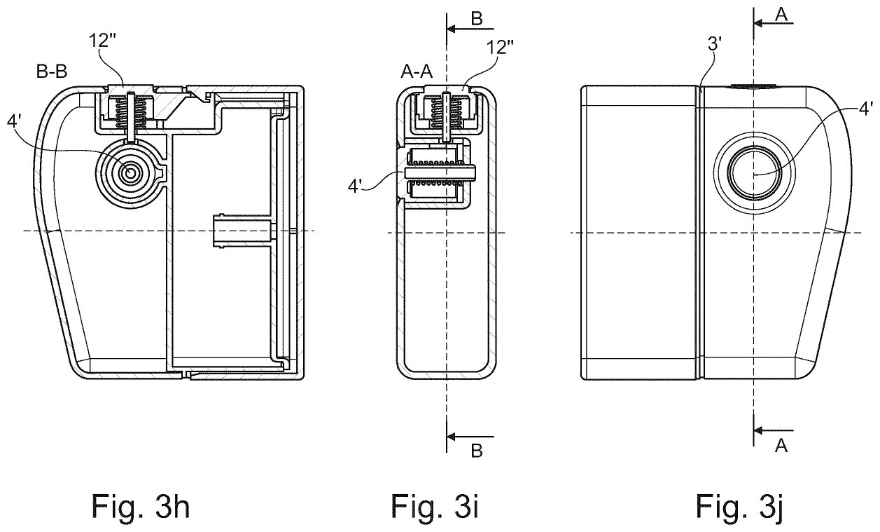

In an alternative embodiment, the actuating element 12 may not be integral with the housing of the accumulator unit 1, but rather configured as a separate part, for example as a push button 12″. Such an embodiment of the actuating element 12 may be realized both in the exemplary embodiment of FIGS. 1 and in the exemplary embodiment of FIGS. 2. An example of such an embodiment is shown in FIGS. 3a-j. FIGS. 3a to 3j show an embodiment with a safety element 4′ that is similar to the safety element 4 in FIGS. 2.

The safety mechanism of FIGS. 3 for releasably locking the accumulator unit 1 to the control unit 2 comprises a latching hook 21, a latching opening 11 and an actuating element 12″. The actuating element 12″ and the latching opening 11 are part of the accumulator unit 1. The latching hook 21 is part of the control unit 2. FIGS. 3b to 3d show the VAD system in a rest position. FIGS. 3e to 3g show the VAD system in the release position.

The latching hook 21 is integral with the housing of the control unit 2. The latching hook has a latching lug 22. The actuating element 12″ is integral with the latching opening 11. In the rest position shown in FIGS. 3b to 3d, the latching hook 21 protrudes through the latching opening 11. The latching lug 22 engages behind an edge of the latching opening 11 so that actuation of the actuating element 12″ is blocked in a direction II. The actuating element 12″ has a spring 122 which pushes the actuating element into the position shown in FIGS. 3b to 3d. To actuate the actuating element 12″, the spring 122 is compressed as shown in FIG. 3f.

The accumulator unit 1 has a safety element 4′. The safety element 4′ may be brought from a safety position into a release position. The safety element 4′ blocks actuation of the actuating element 12″ in the safety position and releases actuation of the actuating element 12″ in the release position. The safety element 4′ is configured as a push button. FIGS. 3b to 3d show the safety element 4 in the safety position. A sleeve 41′ of the safety element 4′ blocks the actuation of the actuating element 12″. The safety element 4′ has a spring 42 that pushes the safety element 4′ into the safety position. To bring the safety element 4′ into the release position, a user must apply a force F to the safety element 4′, which compresses the spring 42 (as shown in FIG. 3f). FIGS. 2e to 2g show the safety element 4 in the release position. A user applies a force F to the safety element so that the sleeve 41′ is displaced in such a way that an opening 411 in the sleeve 41′ is arranged coaxially with respect to a pin 123 of the actuating element 12″. The pin 123 of the actuating element 12″ may then be moved into the opening of the safety element 4′ when the safety element 4′ is actuated, so that the safety element 4′ releases actuation of the actuating element 12″.

Starting from the rest position shown in FIGS. 3b to 3d, the accumulator unit 1 and the control unit 2 were pressed together along the axis a in order to move the accumulator unit 1 or the control unit 2 into a pre-release position. In the pre-release position, the latching hook 21 furthermore protrudes through the latching opening 11. However, the latching lug 22 no longer engages behind the edge of the latching opening 11.

To bring the accumulator unit 1 and control unit into the release position (shown in FIGS. 3e to 3g), the accumulator unit 1 and control unit 2 must first be pressed together so that they are in the pre-release position. The safety element 4′ is then actuated so that actuation of the actuating element 12″ is released. The actuating element 12″ is then actuated, which moves the latching opening downwards so that the latching hook 21 is outside the latching opening 11 (cf. FIGS. 3e to 3g). The accumulator unit 1 and the control unit 2 may now be released from each other.

FIG. 3h shows a sectional view BB, FIG. 3i a sectional view AA, and FIG. 3j a plan view of a control unit 2 and an accumulator unit 1, which correspond to those of FIGS. 3a-g, wherein an elastic element 3′ in the form of a circumferential elastic sealing element, for example a sponge rubber ring, is arranged between the control unit 2 and the accumulator unit 1 instead of a spring 3. The further features described above in relation to the previous figures therefore also apply to the VAD system shown in FIGS. 3h-j. FIG. 3h-j show the control unit 2 and the accumulator unit 1 in an assembled and locked state in the rest position.

Features described in relation to one of the exemplary embodiments may be realized in other exemplary embodiments in addition to or as an alternative to other features. The exemplary embodiments described above are not restrictive. A person skilled in the art will recognize that the features described may be combined in other ways.

To clarify the use of and to hereby provide notice to the public, the phrases “at least one of <A>, <B>, . . . and <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one or more element alone or the one or more element in combination with one or more of the other elements which may also include, in combination, additional elements not listed. Unless otherwise indicated or the context suggests otherwise, as used herein, “a” or “an” means “at least one” or “one or more.”

1. A safety mechanism for locking and releasing a first unit with a second unit, the safety mechanism comprising:
a latching hook having a latching lug, a latching opening and a first actuating element,
wherein
the latching hook in a rest position engages in the latching opening in such a way that the latching hook projects through the latching opening and the latching lug engages at least partially behind an edge of the latching opening,
the latching hook engages in a pre-release position at least partially in the latching opening in such a way that at least part of the latching hook projects through the latching opening and the latching lug is released from the edge of the latching opening,
the latching hook in a release position is arranged outside of the latching opening,
wherein the actuating element is configured in such a way that actuation of the actuating element moves the latching hook and/or the latching opening from the pre-release position into the release position.

2. The safety mechanism of claim 1, wherein the latching hook and the latching opening are configured in such a way that, in the rest position, the latching hook is locked with the latching opening in a first direction and in a second direction, which is perpendicular to the first direction, and the latching hook is movable in a direction for bringing it into the pre-release position.

3. The safety mechanism of claim 2, wherein the latching hook, in the pre-release position, engages in the latching opening in such a way that the latching hook is locked with the latching opening in the first direction and is released in the second direction.

4. The safety mechanism of claim 1, characterized in that wherein actuation of the actuating element is blocked by the latching lug in the rest position.

5. The safety mechanism of claim 1, wherein the actuating element is configured as a pressure element in such a way that a pressure force exerted on the actuating element moves the latching hook and/or the latching opening in such a way that the latching hook is brought from the pre-release position into the release position.

6. The safety mechanism of claim 1, wherein the actuating element is formed integrally with the latching hook or integrally with the latching opening.

7. The safety mechanism of claim 1, comprising an elastic element which is configured and arranged in such a way that it urges the latching hook from the pre-release position into the rest position.

8. The safety mechanism of claim 1, comprising a second latching hook, a second latching opening and a second actuating element, which are arranged mirror-symmetrically to the first latching hook, to the first latching opening and to the first actuating element.

9. The safety mechanism of claim 8, wherein the actuating elements are arranged oppositely such that simultaneous pressing the actuating elements together causes the latching hooks to be in the release position.

10. The safety mechanism of claim 1, comprising a safety element movable from a safety position into a release position, wherein the safety element blocks actuation of the actuating element in the safety position and releases actuation of the actuating element in the release position.

11. A safety system comprising a first unit and a second unit, wherein the first unit is releasably connectable to the second unit via a safety mechanism of claim 1, wherein the latching hook is fixedly connected to the first unit and the latching opening is fixedly connected to the second unit.

12. The safety system of claim 11, wherein the first unit and the second unit are locked together in the rest position and in the pre-release position via the safety mechanism and are released from each other in the release position.

13. A VAD system comprising a safety system of claim 11, wherein one of the first unit and the second unit is a control unit and the other of the second unit and the first unit is an accumulator unit.

14. A method for releasing a first unit from a second unit of a safety system of claim 11, said method comprising the steps of:
I. moving the first unit in the direction of the second unit,
II. actuating the actuating element,
III. releasing the first unit from the second unit,
wherein step II follows step I and step III follows step II to release the first unit from the second unit.

15. The method of claim 14, wherein a safety element is moved between steps I and II from a safety position into a release position, wherein the safety element blocks actuation of the actuating element in the safety position and releases actuation of the actuating element in the release position.

16. The safety mechanism of claim 1, wherein one of the first unit and the second unit is an accumulator unit of a VD system and the other of the second unit and the first unit is a control unit of a VAD system.

17. A method for releasing a first unit from a second unit of a VAD system of claim 13, said method comprising the steps of:
I. moving the first unit in the direction of the second unit,
II. actuating the actuating element,
III. releasing the first unit from the second unit,
III. wherein step II follows step I and step III follows step II to release the first unit from the second unit.

18. The method of claim 14, wherein a safety element is moved between steps I and II from a safety position into a release position, wherein the safety element blocks actuation of the actuating element in the safety position and releases actuation of the actuating element in the release position.
